(19) United States
(12) Patent Application Publication
(10) Pub. No.: US 2025/0235079 A1
(43) Pub. Date: Jul. 24, 2025

(54) MEDICAL SUPPORT DEVICE, ENDOSCOPE, MEDICAL SUPPORT METHOD, AND PROGRAM

(71) Applicant: FUJIFILM Corporation, Tokyo (JP)

(72) Inventor: Toshihiro USUDA, Kanagawa (JP)

(73) Assignee: FUJIFILM Corporation, Tokyo (JP)

(21) Appl. No.: 19/180,154

(22) Filed: Apr. 16, 2025

Related U.S. Application Data
(63) Continuation of application No. PCT/JP2023/036268, filed on Oct. 4, 2023.

(30) Foreign Application Priority Data
Nov. 4, 2022 (JP) 2022-177612

(51) Int. Cl. A61B 1/00; G06V 10/764; G06V 20/50; G16H 10/60; G16H 30/40

(52) U.S. Cl. CPC A61B 1/000094; A61B 1/000096; A61B 1/0005; G06V 10/764; G06V 20/50; G16H 10/60; G16H 30/40; G06V 2201/031

(57) ABSTRACT

Provided is a medical support device including a processor. The processor is configured to specify a papilla type, which is a type of a duodenal papilla, by executing image recognition processing on an intestinal wall image obtained by imaging an intestinal wall including the duodenal papilla in a duodenum with a camera provided in an endoscope scope; and output related information related to the papilla type.

### CROSS-REFERENCE TO RELATED APPLICATIONS

This application is a continuation application of International Application No. PCT/JP2023/036268, filed Oct. 4, 2023, the disclosure of which is incorporated herein by reference in its entirety. Further, this application claims priority from Japanese Patent Application No. 2022-177612, filed Nov. 4, 2022, the disclosure of which is incorporated herein by reference in its entirety.

### BACKGROUND

### 1. Technical Field

The technology of the present disclosure relates to a medical support device, an endoscope, a medical support method, and a program.

### 2. Related Art

JP2020-62218A discloses a learning apparatus comprising an acquisition unit that acquires a plurality of pieces of information in which an image of a duodenal Vater's papilla of a bile duct and information indicating a cannulation method, which is a method of inserting a catheter into the bile duct, are associated with each other; a learning unit that performs machine learning using the information indicating the cannulation method as training data based on the image of the duodenal Vater's papilla of the bile duct; and a storage unit that stores a result of the machine learning performed by the learning unit and the information indicating the cannulation method in association with each other.

### SUMMARY

One embodiment according to the technology of the present disclosure provides a medical support device, an endoscope, a medical support method, and a program that can support implementation of medical care according to the type of a duodenal papilla.

A first aspect according to the technology of the present disclosure is a medical support device comprising a processor, in which the processor is configured to specify a papilla type, which is a type of a duodenal papilla, by executing image recognition processing on an intestinal wall image obtained by imaging an intestinal wall including the duodenal papilla in a duodenum with a camera provided in an endoscope scope; and output related information related to the

A second aspect according to the technology of the present disclosure is the medical support device according to the first aspect, in which the outputting of the related information is displaying the related information on a screen.

A third aspect according to the technology of the present disclosure is the medical support device according to the first aspect or the second aspect, in which the related information includes a schema determined according to the papilla type.

A fourth aspect according to the technology of the present disclosure is the medical support device according to any one of the first to third aspects, in which the related information includes merging format information, and the merging format information is information that is determined according to the papilla type and that is capable of specifying a merging format in which a bile duct and a pancreatic duct merge with each other.

A fifth aspect according to the technology of the present disclosure is the medical support device according to any one of the first to fourth aspects, in which the image recognition processing includes classification processing of classifying the papilla types, and the related information includes degree-of-certainty information indicating a degree of certainty for each papilla type classified by the classification processing.

A sixth aspect according to the technology of the present disclosure is the medical support device according to any one of the first to fifth aspects, in which an appearance frequency of a merging format in which a bile duct and a pancreatic duct merge with each other is determined for each papilla type, and the processor is configured to output, as the related information, information including appearance frequency information indicating the appearance frequency according to the specified papilla type.

A seventh aspect according to the technology of the present disclosure is the medical support device according to any one of the first to fifth aspects, in which the papilla type includes a first papilla type, the first papilla type has any one of a plurality of merging formats in which a bile duct and a pancreatic duct merge with each other, and the processor is configured to output, in a case where the first papilla type is specified as the papilla type, information including appearance frequency information indicating an appearance frequency for each merging format as the related information.

An eighth aspect according to the technology of the present disclosure is the medical support device according to the seventh aspect, in which the first papilla type is a villous type or a flat type, and the plurality of merging formats are a septal type and a common duct type.

A ninth aspect according to the technology of the present disclosure is the medical support device according to any one of the first to eighth aspects, in which the related information includes assistance information, and the assistance information is information for assisting with a medical treatment performed for a merging format in which a bile duct and a pancreatic duct merge with each other and which is determined according to the papilla type.

A tenth aspect according to the technology of the present disclosure is the medical support device according to the ninth aspect, in which the processor is configured to output the assistance information in a case where a plurality of the merging formats are present in the specified papilla type.

An eleventh aspect according to the technology of the present disclosure is the medical support device according to any one of the first to tenth aspects, in which the processor is configured to specify the papilla type by executing the image recognition processing on the intestinal wall image in units of frames.

A twelfth aspect according to the technology of the present disclosure is the medical support device according to any one of the first to tenth aspects, in which the image recognition processing includes first image recognition processing and second image recognition processing, and the processor is configured to detect a duodenal papilla region by executing the first image recognition processing on the intestinal wall image; and specify the papilla type by executing the second image recognition processing on the detected duodenal papilla region.

A thirteenth aspect according to the technology of the present disclosure is the medical support device according to any one of the first to twelfth aspects, in which the related information is stored in an external device and/or a medical record.

A fourteenth aspect of the technology of the present disclosure is an endoscope comprising the medical support device according to any one of the first to thirteenth aspects; and the endoscope scope.

A fifteenth aspect according to the technology of the present disclosure is a medical support method comprising specifying a papilla type, which is a type of a duodenal papilla, by executing image recognition processing on an intestinal wall image obtained by imaging an intestinal wall including the duodenal papilla in a duodenum with a camera provided in an endoscope scope; and outputting related information related to the papilla type.

A sixteenth aspect according to the technology of the present disclosure is a program causing a computer to execute processing comprising specifying a papilla type, which is a type of a duodenal papilla, by executing image recognition processing on an intestinal wall image obtained by imaging an intestinal wall including the duodenal papilla in a duodenum with a camera provided in an endoscope scope; and outputting related information related to the

### BRIEF DESCRIPTION OF THE DRAWINGS

Exemplary embodiments according to the technique of the present disclosure will be described in detail based on the following figures, wherein:

FIG. 1 is a conceptual diagram showing an example of an aspect in which a duodenoscope system is used;

FIG. 2 is a conceptual diagram showing an example of an overall configuration of the duodenoscope system;

FIG. 3 is a block diagram showing an example of a hardware configuration of an electrical system of the duodenoscope system;

FIG. 4 is a conceptual diagram showing an example of an aspect in which a duodenoscope is used;

FIG. 5 is a block diagram showing an example of a hardware configuration of an electrical system of an image processing device;

FIG. 6 is a conceptual diagram showing an example of a correlation between an endoscope scope, an NVM, an image acquisition unit, an image recognition unit, and a support information acquisition unit;

FIG. 7 is a conceptual diagram showing an example of a correlation between a display device, the image acquisition unit, the image recognition unit, the support information acquisition unit, and a display control unit;

FIG. 8 is a flowchart showing an example of a flow of medical support processing;

FIG. 9 is a conceptual diagram showing an example of the correlation between the endoscope scope, the NVM, the image acquisition unit, the image recognition unit, and the support information acquisition unit;

FIG. 10 is a conceptual diagram showing an example of the correlation between the display device, the image acquisition unit, the image recognition unit, the support information acquisition unit, and the display control unit;

FIG. 11 is a conceptual diagram showing an example of the correlation between the endoscope scope, the NVM, the image acquisition unit, the image recognition unit, and the support information acquisition unit;

FIG. 12 is a conceptual diagram showing an example of the correlation between the display device, the image acquisition unit, the image recognition unit, the support information acquisition unit, and the display control unit;

FIG. 13 is a conceptual diagram showing an example of the correlation between the display device, the image acquisition unit, the image recognition unit, the support information acquisition unit, and the display control unit;

FIG. 14 is a conceptual diagram showing an example of the correlation between the endoscope scope, the NVM, the image acquisition unit, the image recognition unit, and the support information acquisition unit; and

FIG. 15 is a conceptual diagram showing an example of an aspect in which an intestinal wall image, support information, and papilla type information generated by the duodenoscope system are stored in an electronic medical record server.

### DETAILED DESCRIPTION

Hereinafter, examples of embodiments of a medical support device, an endoscope, a medical support method, and a program according to the technology of the present disclosure will be described with reference to the accompanying drawings.

First, terms used in the following description will be described.

CPU is an abbreviation for “central processing unit”. GPU is an abbreviation for “graphics processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. I/F is an abbreviation for “interface”. FIFO is an abbreviation for “first in, first out”. ERCP is an abbreviation for “endoscopic retrograde cholangio-pancreatography”.

### First Embodiment

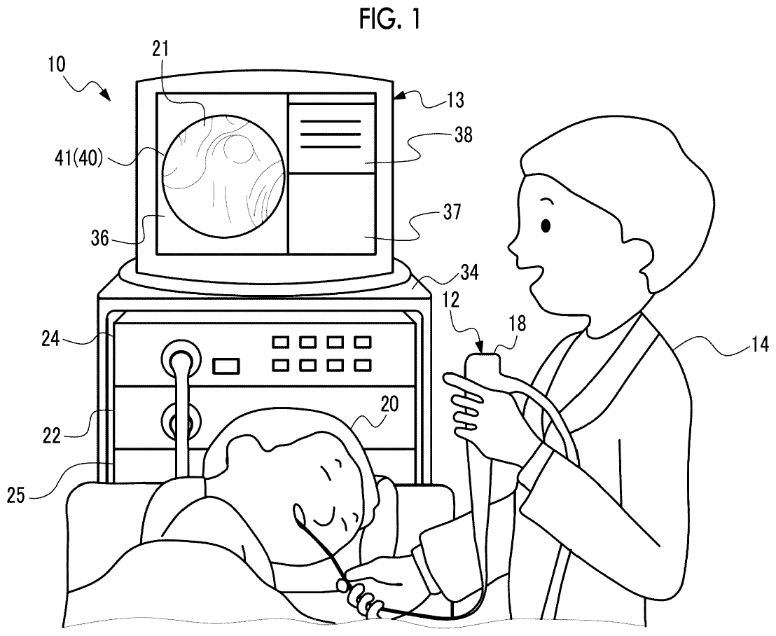

For example, as shown in FIG. 1, a duodenoscope system 10 comprises a duodenoscope 12 and a display device 13. The duodenoscope 12 is used by a doctor 14 in endoscopy. The duodenoscope 12 is communicably connected to a communication device (not shown), and information obtained by the duodenoscope 12 is transmitted to the communication device. The communication device receives the information transmitted from the duodenoscope 12 and performs processing using the received information (for example, the processing of recording the information on an electronic medical record or the like).

The duodenoscope 12 comprises an endoscope scope 18. The duodenoscope 12 is a device for performing medical care on an observation target 21 (for example, a duodenum) included in a body of a subject 20 (for example, a patient) using the endoscope scope 18. The observation target 21 is a target observed by the doctor 14. The endoscope scope 18 is inserted into the body of the subject 20. The duodenoscope 12 causes the endoscope scope 18 inserted into the body of the subject 20 to image the observation target 21 inside the body of the subject 20, and performs various medical treatments on the observation target 21 as necessary. The duodenoscope 12 is an example of the “endoscope” according to the technology of the present disclosure.

The duodenoscope 12 images the inside of the body of the subject 20 to acquire an image showing an aspect of the inside of the body and outputs the image. In the present embodiment, the duodenoscope 12 is an endoscope having an optical imaging function of irradiating the inside of the body with light to image the light reflected by the observation target 21.

The duodenoscope 12 comprises a control device 22, a light source device 24, and an image processing device 25. The control device 22 and the light source device 24 are installed in a wagon 34. A plurality of tables are provided in the wagon 34 in a vertical direction, and the image processing device 25, the control device 22, and the light source device 24 are installed from a lower table to an upper table. In addition, the display device 13 is installed on the uppermost table in the wagon 34.

The control device 22 is a device that controls the entire duodenoscope 12. In addition, the image processing device 25 is a device that performs image processing on the image captured by the duodenoscope 12 under the control of the control device 22.

The display device 13 displays various types of information including an image (for example, an image subjected to image processing by the image processing device 25). An example of the display device 13 is a liquid-crystal display or an EL display. In addition, a tablet terminal with a display may be used instead of the display device 13 or together with the display device 13.

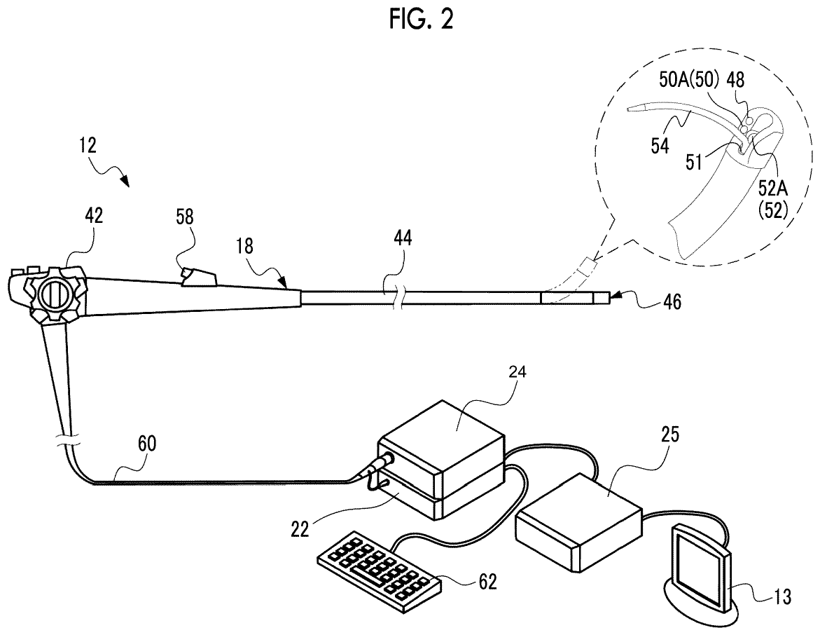

A plurality of screens are displayed side by side on the display device 13. In the example shown in FIG. 1, screens 36, 37, and 38 are shown. An endoscopic image 40 obtained by the duodenoscope 12 is displayed on the screen 36. The observation target 21 is included in the endoscopic image 40. The endoscopic image 40 is an image obtained by imaging the observation target 21 with a camera 48 (see FIG. 2) provided in the endoscope scope 18 inside the body of the subject 20. An example of the observation target 21 includes an intestinal wall of a duodenum. In the following, for convenience of description, an intestinal wall image 41 which is an endoscopic image 40 in which the intestinal wall of the duodenum is imaged is described as an example of the observation target 21. In addition, the duodenum is merely an example, and any region that can be imaged by the duodenoscope 12 may be used. For example, an esophagus or a stomach is given as an example of the region that can be imaged by the duodenoscope 12. The intestinal wall image 41 is an example of an “intestinal wall image” according to the technology of the present disclosure.

A moving image including a plurality of frames of the intestinal wall images 41 is displayed on the screen 36. That is, the plurality of frames of intestinal wall images 41 are displayed on the screen 36 at a predetermined frame rate (for example, several tens of frames/sec).

As shown in FIG. 2 as an example, the duodenoscope 12 comprises an operation part 42 and an insertion part 44. The insertion part 44 is partially bent by operating the operation part 42. The insertion part 44 is inserted while being bent according to the shape of the observation target 21 (for example, the shape of the stomach) in response to the operation of the operation part 42 by the doctor 14.

The camera 48, an illumination device 50, a treatment opening 51, and an elevating mechanism 52 are provided at a distal end part 46 of the insertion part 44. The camera 48 and the illumination device 50 are provided on a side surface of the distal end part 46. That is, the duodenoscope 12 serves as a side-viewing scope. Accordingly, the intestinal wall of the duodenum is easily observed.

The camera 48 is a device that acquires the intestinal wall image 41 as a medical image by imaging the inside of the body of the subject 20. An example of the camera 48 includes a CMOS camera. However, this is merely an example, and the camera 48 may be other types of cameras such as CCD cameras. The camera 48 is an example of the “camera” according to the technology of the present disclosure.

The illumination device 50 has an illumination window 50A. The illumination device 50 emits light through the illumination window 50A. Examples of the type of the light emitted from the illumination device 50 include visible light (for example, white light) and invisible light (for example, near-infrared light). In addition, the illumination device 50 emits special light through the illumination window 50A. Examples of the special light include light for BLI and/or light for LCI. The camera 48 images the inside of the body of the subject 20 using an optical method in a state in which the inside of the body of the subject 20 is irradiated with light by the illumination device 50.

The treatment opening 51 is used as a treatment tool protruding port through which a treatment tool 54 is made to protrude from the distal end part 46, a suction port for suctioning, for example, blood and internal filth, and a delivery port for sending out a fluid.

The treatment tool 54 protrudes from the treatment opening 51 in response to the operation of the doctor 14. The treatment tool 54 is inserted into the insertion part 44 from a treatment tool insertion port 58. The treatment tool 54 passes through the inside of the insertion part 44 through the treatment tool insertion port 58 and protrudes from the treatment opening 51 into the body of the subject 20. In the example shown in FIG. 2, a cannula protrudes from the treatment opening 51 as the treatment tool 54. The cannula is merely an example of the treatment tool 54, and other examples of the treatment tool 54 include a papillotomy knife and a snare.

The elevating mechanism 52 changes a protruding direction of the treatment tool 54 protruding from the treatment opening 51. The elevating mechanism 52 comprises a guide 52A, and the guide 52A rises with respect to the protruding direction of the treatment tool 54, so that the protruding direction of the treatment tool 54 is changed along the guide 52A. Accordingly, it is easy to protrude the treatment tool 54 toward the intestinal wall. In the example shown in FIG. 2, the protruding direction of the treatment tool 54 is changed to a direction perpendicular to a traveling direction of the distal end part 46 by the elevating mechanism 52. The elevating mechanism 52 is operated by the doctor 14 using the operation part 42. Accordingly, the degree of change in the protruding direction of the treatment tool 54 is adjusted.

The endoscope scope 18 is connected to the control device 22 and the light source device 24 via a universal cord 60. The display device 13 and a receiving device 62 are connected to the control device 22. The receiving device 62 receives an instruction from a user (for example, the doctor 14) and outputs the received instruction as an electric signal. In the example shown in FIG. 2, a keyboard is given as an example of the receiving device 62. However, this is merely an example, and the receiving device 62 may be, for example, a mouse, a touch panel, a foot switch and/or a microphone.

The control device 22 controls the entire duodenoscope 12. For example, the control device 22 controls the light source device 24 or transmits and receives various signals to and from the camera 48. The light source device 24 emits light under the control of the control device 22 and supplies the light to the illumination device 50. A light guide is provided in the illumination device 50, and the light supplied from the light source device 24 is emitted from the illumination windows 50A and 50B through the light guide. The control device 22 causes the camera 48 to execute the imaging, acquires the intestinal wall image 41 (see FIG. 1) from the camera 48, and outputs the intestinal wall image 41 to a predetermined output destination (for example, the image processing device 25).

The image processing device 25 is communicably connected to the control device 22, and the image processing device 25 performs image processing on the intestinal wall image 41 output from the control device 22. Details of the image processing in the image processing device 25 will be described below. The image processing device 25 outputs the intestinal wall image 41 subjected to the image processing to a predetermined output destination (for example, the display device 13). In addition, here, the form example in which the intestinal wall image 41 output from the control device 22 is output to the display device 13 via the image processing device 25 has been described, but this is merely an example. The control device 22 and the display device 13 may be connected to each other, and the intestinal wall image 41 subjected to the image processing by the image processing device 25 may be displayed on the display device 13 via the control device 22.

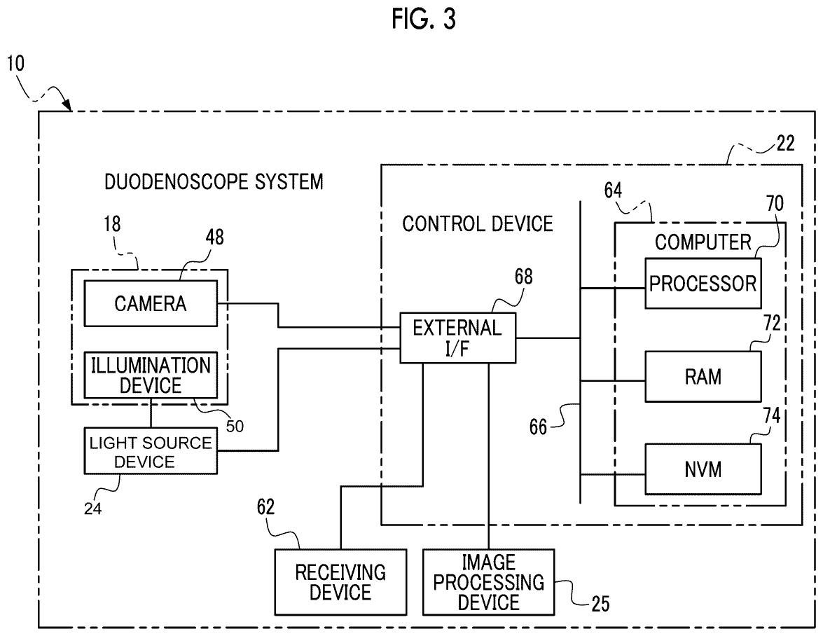

As shown in FIG. 3 as an example, the control device 22 comprises a computer 64, a bus 66, and an external I/F 68. The computer 64 comprises a processor 70, a RAM 72, and an NVM 74. The processor 70, the RAM 72, the NVM 74, and the external I/F 68 are connected to the bus 66.

For example, the processor 70 includes a CPU and a GPU and controls the entire control device 22. The GPU operates under the control of the CPU and is in charge of, for example, executing various processing operations of a graphics system and performing calculation using a neural network. In addition, the processor 70 may be one or more CPUs with which the functions of the GPU have been integrated or may be one or more CPUs with which the functions of the GPU have not been integrated.

The RAM 72 is a memory that temporarily stores information and is used as a work memory by the processor 70. The NVM 74 is a non-volatile storage device that stores, for example, various programs and various parameters. An example of the NVM 74 includes a flash memory (for example, an EEPROM and/or an SSD). In addition, the flash memory is merely an example and may be other non-volatile storage devices, such as HDDs, or a combination of two or more types of non-volatile storage devices.

The external I/F 68 transmits and receives various types of information between a device (hereinafter, also referred to as an “external device”) outside the control device 22 and the processor 70. An example of the external I/F 68 is a USB interface.

The camera 48 is connected to the external I/F 68 as one of the external devices, and the external I/F 68 controls the exchange of various types of information between the camera 48 provided in the endoscope scope 18 and the processor 70. The processor 70 controls the camera 48 via the external I/F 68. In addition, the processor 70 acquires the intestinal wall image 41 (see FIG. 1) obtained by imaging the inside of the body of the subject 20 by the camera 48 provided in the endoscope scope 18 via the external I/F 68.

As one of the external devices, the light source device 24 is connected to the external I/F 68, and the external I/F 68 transmits and receives various types of information between the light source device 24 and the processor 70. The light source device 24 supplies light to the illumination device 50 under the control of the processor 70. The illumination device 50 performs irradiation with the light supplied from the light source device 24.

As one of the external devices, the receiving device 62 is connected to the external I/F 68. The processor 70 acquires the instruction received by the receiving device 62 via the external I/F 68 and executes the processing corresponding to the acquired instruction.

The image processing device 25 is connected to the external I/F 68 as one of the external devices, and the processor 70 outputs the intestinal wall image 41 to the image processing device 25 via the external I/F 68.

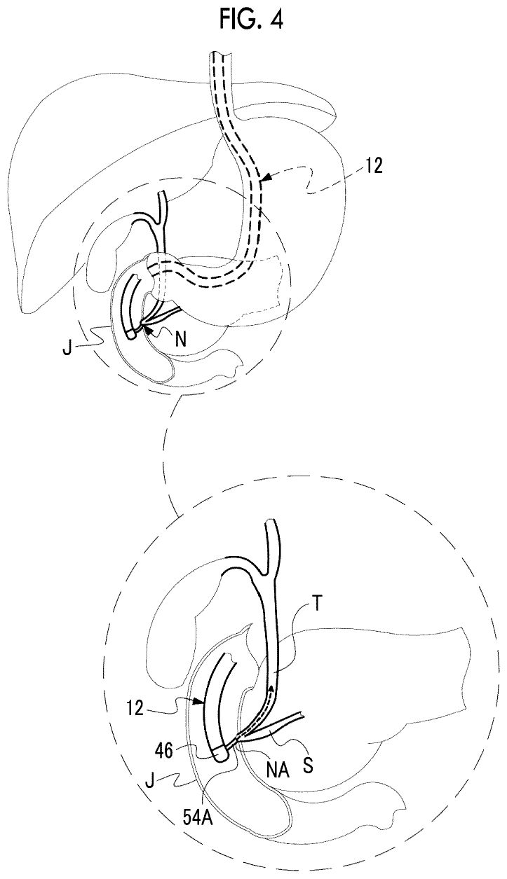

During the treatment on the duodenum using the endoscope, a treatment called endoscopic retrograde cholangio-pancreatography (ERCP) examination may be performed. As shown in FIG. 4 as an example, in the ERCP examination, for example, first, the duodenoscope 12 is inserted into a duodenum J through the esophagus and the stomach. In this case, the insertion state of the duodenoscope 12 may be confirmed by X-ray imaging. Then, the distal end part 46 of the duodenoscope 12 reaches the vicinity of a duodenal papilla N (hereinafter, also simply referred to as a “papilla N”) present in the intestinal wall of the duodenum J.

In the ERCP examination, for example, a cannula 54A is inserted from the papilla N. Here, the papilla N is a part that protrudes from the intestinal wall of the duodenum J, and an opening of an end part of a bile duct T (for example, a common bile duct, an intrahepatic bile duct, or a cystic duct) and a pancreatic duct S are present in a papillary protuberance NA of the papilla N. X-ray imaging is performed in a state in which a contrast agent is injected into the bile duct T, the pancreatic duct S, and the like through the cannula 54A from the opening of the papilla N. In the ERCP examination, it is important to perform a treatment after grasping the type of the papilla N. This is because, in a case where the cannula 54A is inserted, the type of the papilla N affects the success or failure of the insertion, and the state (for example, the shape of a duct) of the bile duct T and the pancreatic duct S corresponding to the type of the papilla N affects the success or failure of intubation after the insertion. However, for example, since the doctor 14 operates the duodenoscope 12, it is difficult to always grasp the type of the papilla N and the like.

In addition, for example, in a case of the doctor 14 who has little experience in the ERCP examination, information related to a procedure including the type of the papilla N may be referred to. However, in this case, it is difficult to check the information related to the procedure by referring to a text or a memo because the doctor 14 is also concentrating on the operation of the duodenoscope 12.

Thus, in view of such circumstances, in the present embodiment, the medical support processing is performed by a processor 82 of the image processing device 25 in order to support the implementation of the medical care according to the type of the duodenal papilla.

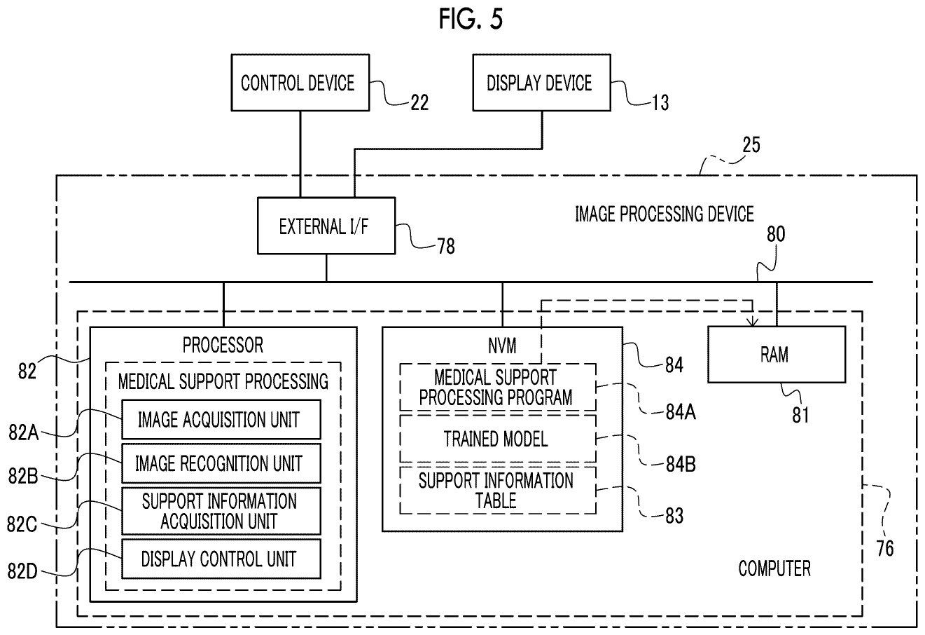

As shown in FIG. 5 as an example, the image processing device 25 comprises a computer 76, an external I/F 78, and a bus 80. The computer 76 comprises the processor 82, an NVM 84, and a RAM 81. The processor 82, the NVM 84, the RAM 81, and the external I/F 78 are connected to the bus 80. The computer 76 is an example of the “medical support device” and the “computer” according to the technology of the present disclosure. The processor 82 is an example of the “processor” according to the technology of the present disclosure.

In addition, a hardware configuration (that is, the processor 82, the NVM 84, and the RAM 81) of the computer 76 is essentially the same as a hardware configuration of the computer 64 shown in FIG. 3. Thus, the description of the hardware configuration of the computer 76 will be omitted here. In addition, since the role of the external I/F 78 in the image processing device 25 to transmit and receive information to and from the outside is essentially the same as the role performed by the external I/F 68 in the control device 22 shown in FIG. 3, the description thereof will be omitted here.

A medical support processing program 84A is stored in the NVM 84. The medical support processing program 84A is an example of the “program” according to the technology of the present disclosure. The processor 82 reads the medical support processing program 84A from the NVM 84 and executes the read medical support processing program 84A on the RAM 81. The medical support processing is realized by the processor 82 operating as an image acquisition unit 82A, an image recognition unit 82B, a support information acquisition unit 82C, and a display control unit 82D according to the medical support processing program 84A executed on the RAM 81.

A trained model 84B is stored in the NVM 84. In the present embodiment, the image recognition unit 82B performs AI-type image recognition processing as the image recognition processing for object detection. The trained model 84B is optimized by performing machine learning on the neural network in advance.

A support information table 83 is stored in the NVM 84. Details of the support information table 83 will be described below.

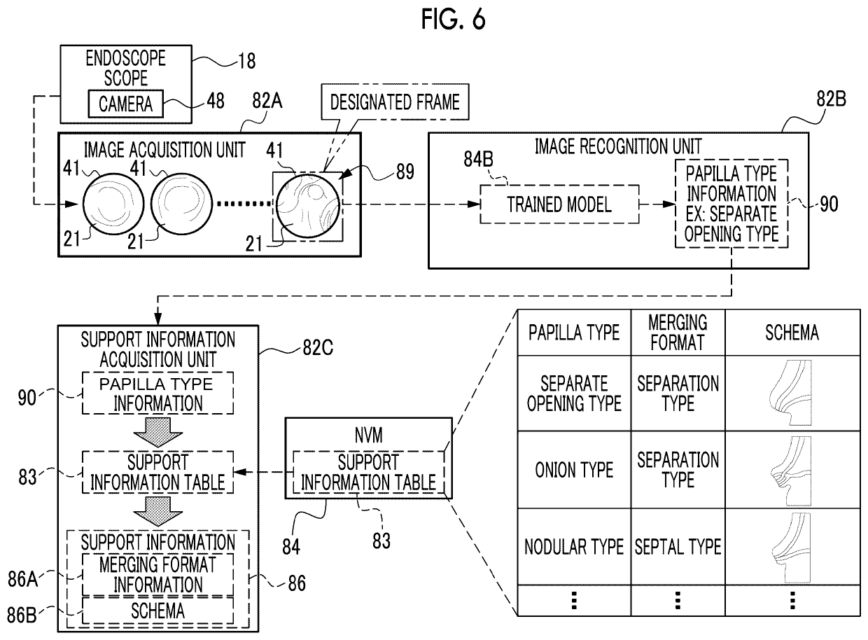

As shown in FIG. 6 as an example, the image acquisition unit 82A acquires the intestinal wall image 41, which is generated by being imaged according to an imaging frame rate (for example, several tens of frames/second) by the camera 48 provided in the endoscope scope 18, from the camera 48 in units of one frame.

The image acquisition unit 82A holds a time-series image group 89. The time-series image group 89 is a plurality of time-series intestinal wall images 41 in which the observation target 21 is captured. The time-series image group 89 includes, for example, a predetermined number of frames (for example, a predetermined number of frames within a range of several tens to several hundreds of frames) of intestinal wall images 41. The image acquisition unit 82A updates the time-series image group 89 using a FIFO method each time the intestinal wall image 41 is acquired from the camera 48.

Here, the form example in which the time-series image group 89 is held and updated by the image acquisition unit 82A has been described, but this is merely an example. For example, the time-series image group 89 may be held and updated in a memory, such as the RAM 81, which is connected to the processor 82.

The image recognition unit 82B acquires the intestinal wall image 41 of a frame designated by the user in the time-series image group 89 held by the image acquisition unit 82A. The designated frame is, for example, a frame at a time point designated by the user by operating the operation part 42. The image recognition unit 82B performs image recognition processing using the trained model 84B on the intestinal wall image 41. By performing the image recognition processing, the type of the papilla N included in the observation target 21 is specified. In the present embodiment, the specification of the type of the papilla N refers to the processing of storing papilla type information 90 (for example, the name of the type of the papilla N captured in the intestinal wall image 41) capable of specifying the type of the papilla N in association with the intestinal wall image 41 in a memory. The papilla type information 90 is an example of the “related information” according to the technology of the present disclosure.

The trained model 84B is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is an annotation capable of specifying the type of the papilla N.

In addition, here, although the form example in which only one trained model 84B is used by the image recognition unit 82B is given as an example, this is merely an example. For example, the trained model 84B selected from a plurality of the trained models 84B may be used by the image recognition unit 82B. In this case, each trained model 84B is created by performing machine learning specialized for each procedure (for example, the position of the duodenoscope 12 with respect to the papilla N, or the like) of the ERCP examination, and the trained model 84B corresponding to the procedure of the ERCP examination currently being performed may be selected and used by the image recognition unit 82B.

The image recognition unit 82B inputs the intestinal wall image 41 acquired from the image acquisition unit 82A to the trained model 84B. Accordingly, the trained model 84B outputs the papilla type information 90 corresponding to the input intestinal wall image 41. The image recognition unit 82B acquires the papilla type information 90 output from the trained model 84B.

The support information acquisition unit 82C acquires support information 86 corresponding to the type of the papilla N. The support information 86 is information provided to the user in order to support the procedure in the ERCP examination. The support information 86 includes merging format information 86A and a schema 86B. The merging format information 86A is information that is determined according to the type of the papilla N and that is capable of specifying a merging format in which the bile duct and the pancreatic duct merge with each other. In addition, the schema 86B is an image showing a state in which the bile duct and the pancreatic duct merge with each other. The support information 86, the merging format information 86A, and the schema 86B are examples of the “related information” according to the technology of the present disclosure. In addition, the merging format information 86A is an example of the “merging format information” according to the technology of the present disclosure, and the schema 86B is an example of the “schema” according to the technology of the present disclosure.

The support information acquisition unit 82C acquires the papilla type information 90 from the image recognition unit 82B. In addition, the support information acquisition unit 82C acquires the support information table 83 from the NVM 84. The support information acquisition unit 82C acquires the support information 86 corresponding to the papilla type information 90 by using the support information table 83. Here, the support information table 83 is information in which the papilla type information 90, the merging format information 86A, and the schema 86B which have a correspondence relationship with each other are associated with each other according to the correspondence relationship. The support information table 83 is, for example, a table in which the papilla type information 90 is used as input information and the merging format information 86A and the schema 86B corresponding to the type of the papilla N are used as output information.

The example shown in FIG. 6 shows an example in which, in the support information table 83, in a case where the type of the papilla Nis a separate opening type, the merging format is a separation type, and the schema 86B is an image showing a state in which the bile duct and the pancreatic duct are separated in the papilla N. In addition, an example is shown in which, in the support information table 83, in a case where the type of the papilla N is an onion type, the merging format is a separation type, and the schema 86B is an image showing a state in which the bile duct and the pancreatic duct are separated in the papilla N and the pancreatic duct is branched in the papilla N. Moreover, an example is shown in which, in the support information table 83, in a case where the type of the papilla N is a nodular type, the merging format is a septal type, and the schema 86B is an image showing a state in which the bile duct and the pancreatic duct are adjacent to each other on a distal end side of a protrusion of the papilla N.

In addition, here, the examples of the separate opening type, the onion type, and the nodular type are given as the input information of the support information table 83, but this is merely an example. The content of the input information and the content of the output information in the support information table 83 are appropriately determined based on medical knowledge related to the type of the papilla N and the merging format. In addition, the output information of the support information table 83 may be only the merging format information 86A. In this case, the schema 86B has the merging format information 86A as accessory information. Then, the support information acquisition unit 82C acquires the schema 86B having the accessory information corresponding to the merging format information 86A based on the merging format information 86A acquired by using the support information table 83.

In addition, in the derivation of the support information 86, a support information calculation expression (not shown) may be used instead of the support information table 83. The support information calculation expression is a calculation expression in which a value indicating the type of the papilla N is set as an independent variable, and a value indicating the merging format and a value indicating the schema 86B are set as dependent variables.

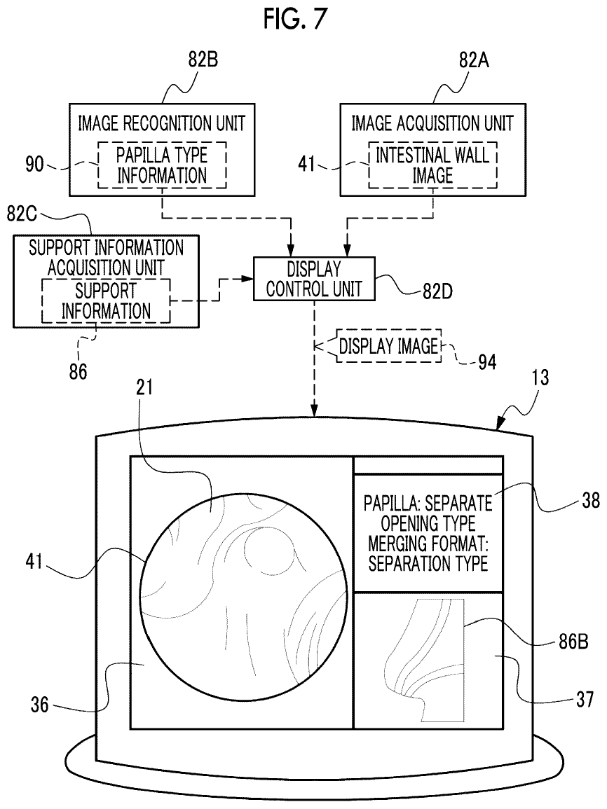

As shown in FIG. 7 as an example, the display control unit 82D acquires the intestinal wall image 41 from the image acquisition unit 82A. In addition, the display control unit 82D acquires the papilla type information 90 from the image recognition unit 82B. Moreover, the display control unit 82D acquires the support information 86 from the support information acquisition unit 82C. The display control unit 82D generates a display image 94 including the intestinal wall image 41, the type of the papilla N indicated by the papilla type information 90, and the merging format and the schema indicated by the support information 86, and outputs the display image 94 to the display device 13. Specifically, the display control unit 82D performs graphical user interface (GUI) control for displaying the display image 94 to cause the display device 13 to display the screens 36 to 38. The screens 36 to 38 are an example of the “screen” according to the technology of the present disclosure.

In the example shown in FIG. 7, the intestinal wall image 41 is displayed on the screen 36. In addition, the schema 86B is displayed on the screen 37. Moreover, a message indicating the type of the papilla N and a message indicating the merging format are displayed on the screen 38. For example, the doctor 14 visually recognizes the intestinal wall image 41 displayed on the screen 36, and further visually recognizes the schema 86B displayed on the screen 37 and a message displayed on the screen 38. Accordingly, it is possible to use information on the type of the papilla N and the merging format in the work of inserting a cannula into the papilla N.

Here, the form example in which the intestinal wall image 41, the papilla type information 90, and the support information 86 are displayed on the screens 36 to 38 of the display device 13 has been described, but this is merely an example. The intestinal wall image 41, the papilla type information 90, and the support information 86 may be displayed on one screen. In addition, the intestinal wall image 41, the papilla type information 90, and the support information 86 may be displayed on separate display devices 13.

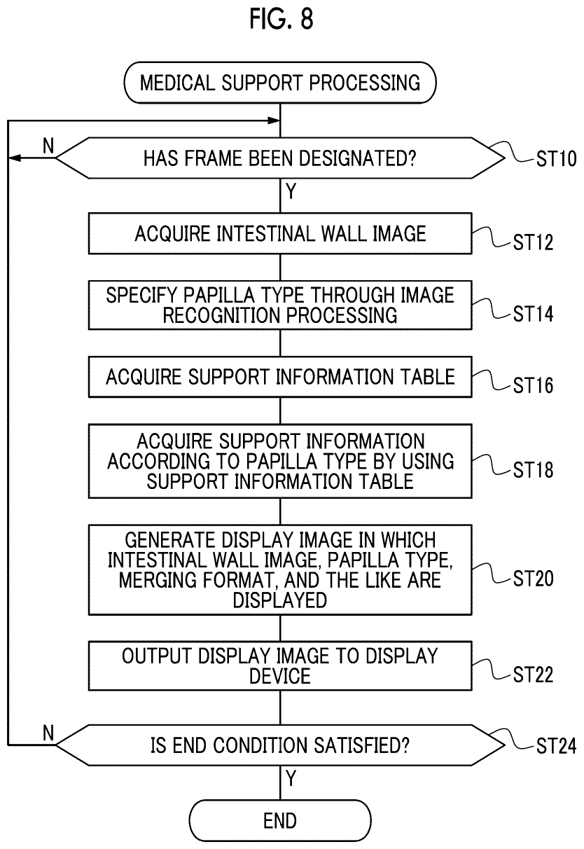

Next, the operation of a portion of the duodenoscope system 10 according to the technology of the present disclosure will be described with reference to FIG. 8.

FIG. 8 shows an example of a flow of the medical support processing performed by the processor 82. The flow of the medical support processing shown in FIG. 8 is an example of the “medical support method” according to the technology of the present disclosure.

In the medical support processing shown in FIG. 8, first, in step ST10, the image acquisition unit 82A determines whether or not the user has designated a frame for the time-series image group 89 captured by the camera 48 provided in the endoscope scope 18. In step ST10, in a case where the frame is not designated, the determination result is “No”, and the determination in step ST10 is made again. In step ST10, in a case where the frame is designated, the determination result is “Yes”, and the medical support processing proceeds to step ST12.

In step ST12, the image acquisition unit 82A acquires the intestinal wall image 41 of the designated frame from the camera 48 provided in the endoscope scope 18. After the processing in step ST12 is executed, the medical support processing proceeds to step ST14.

In step ST14, the image recognition unit 82B performs image recognition processing (that is, image recognition processing using the trained model 84B) using the AI method on the intestinal wall image 41 acquired in step ST12 to detect the type of the papilla N. After the processing in step ST14 is executed, the medical support processing proceeds to step ST16.

In step ST16, the support information acquisition unit 82C acquires the support information table 83 from the NVM 84. After the processing in step ST16 is executed, the medical support processing proceeds to step ST18.

In step ST18, the support information acquisition unit 82C acquires the support information 86 corresponding to the type of the papilla N by using the support information table 83. Specifically, the support information acquisition unit 82C acquires the merging format information 86A and the schema 86B as the support information 86 from the support information table 83. After the processing in step ST18 is executed, the medical support processing proceeds to step ST20.

In step ST20, the display control unit 82D generates a display image 94 in which the intestinal wall image 41, the type of the papilla N indicated by the papilla type information 90, the merging format indicated by the merging format information 86A, and the schema 86B are displayed. After the processing in step ST20 is executed, the medical support processing proceeds to step ST22.

In step ST22, the display control unit 82D outputs the display image 94 generated in step ST20 to the display device 13. After the processing in step ST22 is executed, the medical support processing proceeds to step ST24.

In step ST24, the display control unit 82D determines whether or not a condition for ending the medical support processing is satisfied. An example of the medical support processing end condition is a condition (for example, a condition in which an instruction to end the medical support processing is received by the receiving device 62) in which an instruction to end the medical support processing is issued to the duodenoscope system 10.

In a case where a condition to end the medical support processing is not satisfied in step ST24, the determination result is “No”, and the medical support processing proceeds to step ST10. In a case where the condition to end the medical support processing is satisfied in step ST24, the determination result is “Yes”, and the medical support processing ends.

As described above, in the duodenoscope system 10 according to the first embodiment, the type of the papilla N is specified by performing the image recognition processing on the intestinal wall image 41 by the image recognition unit 82B in the processor 82. Then, the support information acquisition unit 82C acquires the support information 86 based on the papilla type information 90. The display control unit 82D outputs the papilla type information 90 and the support information 86 to the outside (for example, the display device 13). Since the type of the papilla N indicated by the papilla type information 90 is displayed together with the intestinal wall image 41 on the display device 13, the user can grasp the type of the papilla N while operating the duodenoscope 12. Accordingly, in the present configuration, it is possible to support the implementation of the medical care according to the type of the papilla N.

In addition, in the duodenoscope system 10 according to the first embodiment, the type of the papilla N indicated by the papilla type information 90, the merging format of the bile duct and the pancreatic duct indicated by the merging format information 86A, and the schema 86B are displayed on the display device 13 under the control of the display control unit 82D. The user can visually recognize various types of information displayed on the display device 13 while operating the duodenoscope 12. Accordingly, in the present configuration, it is possible to visually support the implementation of the medical care according to the type of the papilla N.

In addition, in the duodenoscope system 10 according to the first embodiment, the support information 86 includes the schema 86B that is determined according to the type of the papilla N. For example, the schema 86B is an image schematically showing the merging format of the bile duct and the pancreatic duct. Accordingly, visual support using the schema 86B is realized as support for performing medical care according to the type of the papilla N. In addition, for example, since the schema 86B is included in the support information 86 compared to a case where the support information 86 is displayed in text only, it is possible to easily grasp the information that can be used for the implementation of the medical care.

In addition, in the duodenoscope system 10 according to the first embodiment, the support information 86 includes the merging format information 86A indicating the merging format of the bile duct and the pancreatic duct. The merging format information 86A is information that is determined according to the type of the papilla N and that is capable of specifying the merging format in which the bile duct and the pancreatic duct merge with each other. Accordingly, it is possible to allow the user to recognize the merging format in which the bile duct and the pancreatic duct merge with each other. In the ERCP examination, a treatment tool such as the cannula may be intubated into the bile duct or the pancreatic duct. In this case, the merging format of the bile duct and the pancreatic duct (for example, whether the bile duct and the pancreatic duct are independent ducts or a common duct) affects the success or failure of intubation. Therefore, as the user is made to recognize the merging format of the bile duct and the pancreatic duct, the support for the implementation of the medical care is realized.

In addition, in the duodenoscope system 10 according to the first embodiment, the image recognition unit 82B of the processor 82 performs the image recognition processing in units of frames to specify the type of the papilla N included in the intestinal wall image 41. Accordingly, the specification of the type of the papilla N with a simple configuration is realized, compared to a case where a part of the intestinal wall image 41 is extracted and the image recognition processing is performed in units of the extracted image regions.

### Second Embodiment

In the above first embodiment, although the form example in which the type of the papilla N is specified by the image recognition processing in the image recognition unit 82B has been described, the technology of the present disclosure is not limited to this. In the present second embodiment, as a result of the image recognition processing in the image recognition unit 82B, the type of the papilla N is classified, and the degree of certainty for each classified type of the papilla N is obtained.

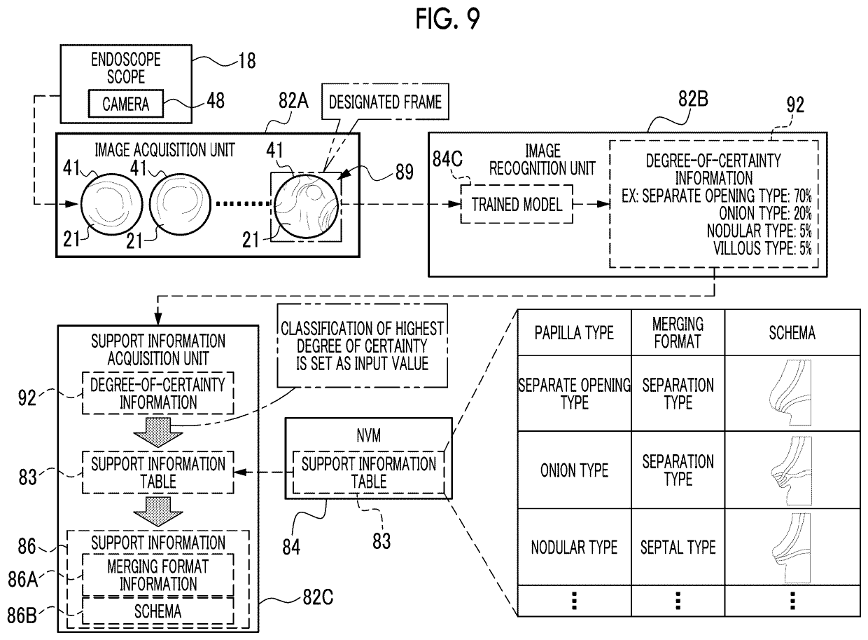

As shown in FIG. 9 as an example, the image acquisition unit 82A acquires the intestinal wall image 41 from the camera 48 provided in the endoscope scope 18. The image recognition unit 82B acquires the intestinal wall image 41 of a frame designated by the user. The image recognition unit 82B performs image recognition processing using a trained model 84C on the intestinal wall image 41. By performing the image recognition processing, the type of the papilla N included in the observation target 21 is classified, and the degree of certainty for each classified type of the papilla N is output. That is, the image recognition processing includes classification processing of classifying the type of the papilla N. As described above, a plurality of types of the papillae determined based on the medical findings are present in the papilla N, and in the classification processing, it is determined which of these types of papillae the papilla N belongs to. Then, in the classification processing, the degree of certainty for each type of the papilla N is calculated according to the classification result of the papilla N. Here, the degree of certainty is a statistical measure indicating the certainty of the classification result. The degree of certainty is, for example, a plurality of scores (scores for each papilla type N) input to an activation function (for example, a softmax function or the like) of an output layer of the trained model 84C.

The trained model 84C is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is a classification result (for example, data in which the type of the papilla N is annotated as a multi-label) of the papilla N.

The image recognition unit 82B inputs the intestinal wall image 41 acquired from the image acquisition unit 82A to the trained model 84C. Accordingly, the trained model 84C outputs degree-of-certainty information 92 corresponding to the input intestinal wall image 41. The image recognition unit 82B acquires the degree-of-certainty information 92 output from the trained model 84B. The degree-of-certainty information 92 includes the degree of certainty for each type of the papilla N in the intestinal wall image 41 in which the papilla Nis captured. The degree-of-certainty information 92 is an example of the “degree-of-certainty information” according to the technology of the present disclosure.

The support information acquisition unit 82C acquires the degree-of-certainty information 92 from the image recognition unit 82B. The support information acquisition unit 82C acquires the support information 86 corresponding to the type of the papilla N indicating the highest degree of certainty among the degrees of certainty indicated by the degree-of-certainty information 92. Specifically, the support information acquisition unit 82C acquires the merging format information 86A and the schema 86B corresponding to the type of the papilla N having the highest degree of certainty by using the support information table 83.

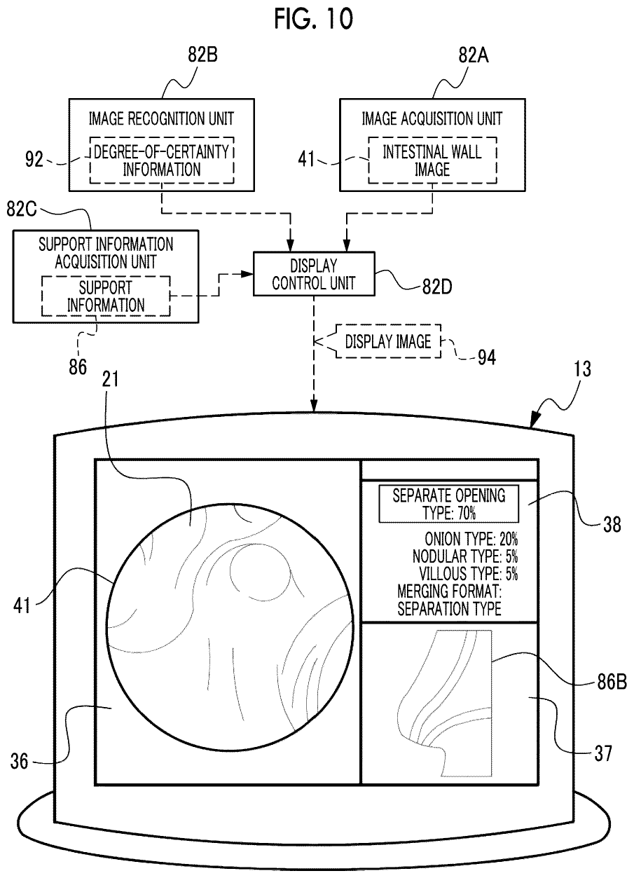

As shown in FIG. 10 as an example, the display control unit 82D acquires the intestinal wall image 41 from the image acquisition unit 82A. In addition, the display control unit 82D acquires the degree-of-certainty information 92 from the image recognition unit 82B. Moreover, the display control unit 82D acquires the support information 86 from the support information acquisition unit 82C. The display control unit 82D generates a display image 94 including the intestinal wall image 41, the degree of certainty for each papilla type N indicated by the degree-of-certainty information 92, and the merging format and the schema indicated by the support information 86, and causes the display device 13 to display the screens 36 to 38.

In the example shown in FIG. 10, the intestinal wall image 41 is displayed on the screen 36, and the schema 86B is displayed on the screen 37. A message indicating the degree of certainty of the papilla N and a message indicating the merging format are displayed on the screen 38. The example shown in FIG. 10 shows an example in which the type and the degree of certainty of the papilla N are the separate opening type: 70%, the onion type: 20%, the nodular type: 5%, and a villous type: 5%. The message of the separate opening type, which is the type of the papilla N with the highest degree of certainty, is surrounded by a frame as a display that can be distinguished from the others.

Here, the form example in which all the classification results are displayed has been described, but this is merely an example. For example, an aspect may be adopted in which only a classification result with a predetermined degree of certainty (for example, 30%) or more is displayed. In addition, the display of the message with the highest degree of certainty may be in another distinguishable aspect, for example, an aspect in which the color or font is changed. In addition, the display of the message having the highest degree of certainty, which is distinguishable from the others, may not be performed.

For example, the doctor 14 visually recognizes the intestinal wall image 41 displayed on the screen 36, and further visually recognizes the schema 86B displayed on the screen 37 and a message displayed on the screen 38. Accordingly, it is possible to use information on the type of the papilla N and the merging format in the work of inserting a cannula into the papilla N.

As described above, in the duodenoscope system 10 according to the present second embodiment, the image recognition processing is performed in the image recognition unit 82B of the processor 82. The image recognition processing includes classification processing of classifying the type of the papilla N. Then, as a result of the image recognition processing on the intestinal wall image 41 by the image recognition unit 82B, the type of the papilla N is classified, and the degree-of-certainty information 92 indicating the degree of certainty for each classified type of the papilla N is output from the image recognition unit 82B. The display device 13 displays the degree of certainty for each type of the papilla N indicated by the degree-of-certainty information 92. The user can grasp the type of the papilla N and the degree of certainty while operating the duodenoscope 12. Accordingly, in a case where the user determines the type of the papilla N, it is possible to reduce the probability of making a wrong determination. That is, the user can grasp the certainty of the specified result and the possibility of the other types of the papilla N, compared to a case where only the result obtained by specifying the type of the papilla N is displayed. Accordingly, in the present configuration, it is possible to support the implementation of the medical care according to the type of the papilla N.

### Third Embodiment

In the above second embodiment, although the form example in which the degree of certainty of the type of the papilla N is displayed has been described, the technology of the present disclosure is not limited to this. In the present third embodiment, the frequency of appearance of the merging format of the bile duct and the pancreatic duct is displayed together with the degree of certainty.

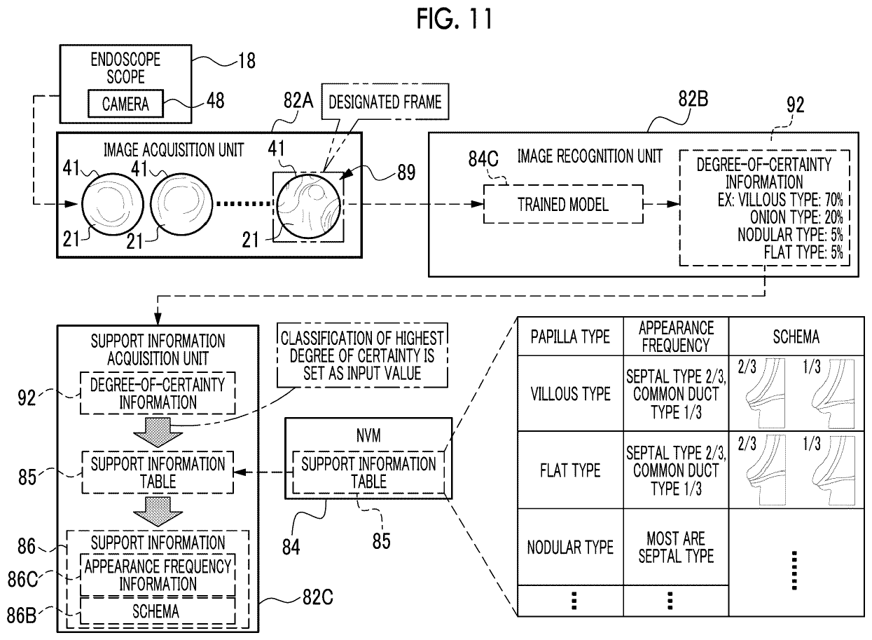

As shown in FIG. 11 as an example, the image acquisition unit 82A acquires the intestinal wall image 41 from the camera 48 provided in the endoscope scope 18. The image recognition unit 82B performs image recognition processing using the trained model 84C on the intestinal wall image 41. The image recognition unit 82B inputs the intestinal wall image 41 acquired from the image acquisition unit 82A to the trained model 84C. Accordingly, the trained model 84C outputs degree-of-certainty information 92 corresponding to the input intestinal wall image 41. The image recognition unit 82B acquires the degree-of-certainty information 92 output from the trained model 84B.

The support information acquisition unit 82C acquires the degree-of-certainty information 92 from the image recognition unit 82B. The support information acquisition unit 82C acquires appearance frequency information 86C and the schema 86B corresponding to the type of the papilla N with the highest degree of certainty by using a support information table 85.

Here, the support information table 85 is a table in which the papilla type information 90, the appearance frequency information 86C, and the schema 86B that have a correspondence relationship with each other are associated with each other according to the correspondence relationship. The support information table 85 is a table in which the type of the papilla N indicated by the papilla type information 90 is set as input information, and the appearance frequency information 86C and the schema 86B corresponding to the type of the papilla N are set as output information.

The example shown in FIG. 11 shows an example in which, in the support information table 85, in a case where the type of the papilla N is the villous type, the appearance frequency of the merging format is ⅔ of the septal type and ⅓ of the common duct type, and the schema 86B is an image showing the septal type and the common duct type. In addition, an example is shown in which, in the support information table 85, in a case where the type of the papilla N is a flat type, the appearance frequency of the merging format is ⅔ of the septal type and ⅓ of the common duct type, and the schema 86B is an image showing the septal type and the common duct type. Moreover, an example is shown in which, in the support information table 85, in a case where the type of the papilla N is the nodular type, the appearance frequency of the merging format is mostly the septal type. Here, the villous type and the flat type are examples of the “first papilla type” according to the technology of the present disclosure.

In addition, here, examples of the villous type, the flat type, and the nodular type are given as the input information of the support information table 85, but this is merely an example. The content of the input information and the content of the output information in the support information table 85 are appropriately determined based on the medical knowledge related to the type of the papilla N and the appearance frequency of the merging format. In addition, the output information of the support information table 85 may be only the appearance frequency information 86C. In this case, the schema 86B has the appearance frequency information 86C as the accessory information. Then, the support information acquisition unit 82C acquires the schema 86B having the accessory information corresponding to the appearance frequency information 86C based on the appearance frequency information 86C acquired by using the support information table 85.

In addition, in the derivation of the support information 86, a support information calculation expression (not shown) may be used instead of the support information table 85. The support information calculation expression is a calculation expression in which the type of the papilla Nis set as an independent variable and the appearance frequency information 86C and the schema 86B are set as dependent variables.

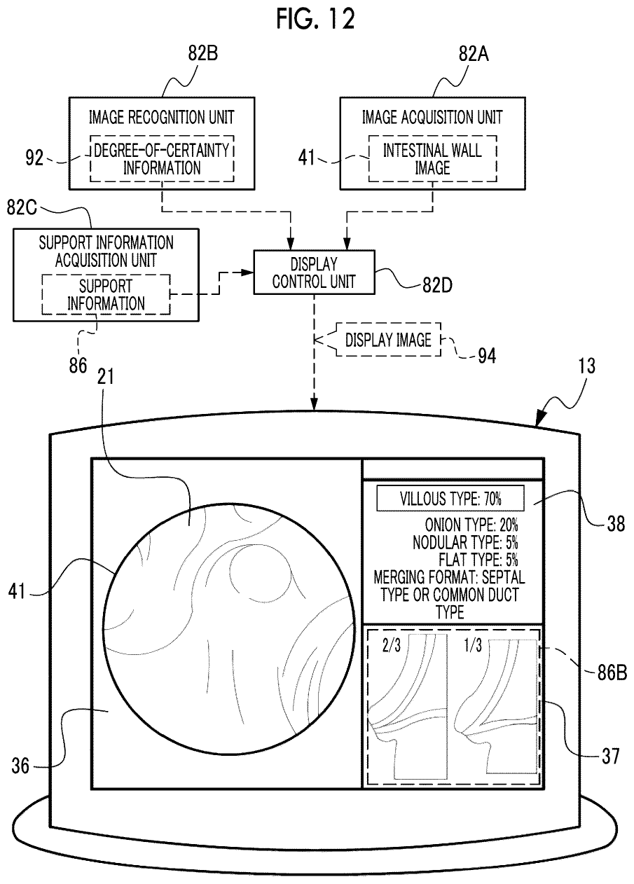

As shown in FIG. 12 as an example, the display control unit 82D acquires the intestinal wall image 41 from the image acquisition unit 82A. In addition, the display control unit 82D acquires the degree-of-certainty information 92 from the image recognition unit 82B. Moreover, the display control unit 82D acquires the support information 86 from the support information acquisition unit 82C. The display control unit 82D generates a display image 94 including the intestinal wall image 41, the degree of certainty for each papilla type N indicated by the degree-of-certainty information 92, and the appearance frequency of the merging format and the schema 86B indicated by the support information 86, and displays the screens 36 to 38 on the display device 13.

In the example shown in FIG. 12, the intestinal wall image 41 is displayed on the screen 36, and the schema 86B is displayed on the screen 37. In the example shown in FIG. 12, the schema 86B includes an image showing a septal type and an image showing a common duct type. In addition, ⅔, which is the appearance frequency, is displayed on the upper left of the image showing the septal type, and ⅓, which is the appearance frequency, is displayed on the upper left of the image showing the common duct type. A message indicating the degree of certainty of the papilla N and a message indicating the merging format are displayed on the screen 38. The message indicating the merging format indicates that the merging format is a septal type or a common duct type.

For example, the doctor 14 visually recognizes the intestinal wall image 41 displayed on the screen 36, and further visually recognizes the schema 86B displayed on the screen 37 and a message displayed on the screen 38. Accordingly, it is possible to use information on the type of the papilla N and the appearance frequency of the merging format in the work of inserting the cannula into the papilla N.

As described above, in the duodenoscope system 10 according to the present third embodiment, in the support information acquisition unit 82C of the processor 82, the appearance frequency information 86C indicating the appearance frequency of the merging format of the bile duct and the pancreatic duct and the schema 86B are acquired by using the support information table 85. Then, the support information acquisition unit 82C outputs the appearance frequency information 86C and the schema 86B as the support information 86. The appearance frequency indicated by the appearance frequency information 86C and the schema 86B are displayed on the display device 13. The user can grasp the type of the papilla N and the appearance frequency of the merging format while operating the duodenoscope 12. Accordingly, in a case where the user visually determines the type of the papilla N, it is possible to contribute to the realization of high-accuracy determination by the user.

In addition, in the duodenoscope system 10 according to the present third embodiment, the appearance frequency information 86C includes information indicating the appearance frequency (for example, ⅔ of the septal type and ⅓ of the common duct type) for each merging format. In addition, the schema 86B shows the appearance frequency together with the image showing the merging format. The support information acquisition unit 82C outputs the appearance frequency information 86C and the schema 86B, and a message indicating the appearance frequency of the merging format of the bile duct and the pancreatic duct and the schema 86B are displayed on the display device 13. The user can grasp the type of the papilla N and the appearance frequency of the merging format while operating the duodenoscope 12. Accordingly, in a case where the user visually determines which of the plurality of merging formats the type of the papilla N has, it is possible to contribute to the realization of the high-accuracy determination by the user.

In addition, in the duodenoscope system 10 according to the present third embodiment, in a case where the type of the papilla N is the villous type or the flat type, the plurality of merging formats are the septal type or the common duct type. For example, an example is shown in which, in the support information table 85, in a case where the type of the papilla N is the villous type, the appearance frequency of the merging format is ⅔ of the septal type and ⅓ of the common duct type, and the schema 86B is an image showing the septal type and the common duct type. In addition, an example is shown in which, in the support information table 85, in a case where the type of the papilla N is a flat type, the appearance frequency of the merging format is ⅔ of the septal type and ⅓ of the common duct type, and the schema 86B is an image showing the septal type and the common duct type. Accordingly, in a case where the user visually determines whether the merging format of the villous type or the flat type of the papilla is the septal type or the common duct type, it is possible to contribute to the realization of the high-accuracy determination by the user.

### First Modification Example

In the above third embodiment, although the form example in which the frequency of appearance of the merging format of the bile duct and the pancreatic duct is displayed has been described, the technology of the present disclosure is not limited to this. In the present first modification example, a message for assisting with the medical treatment is displayed.

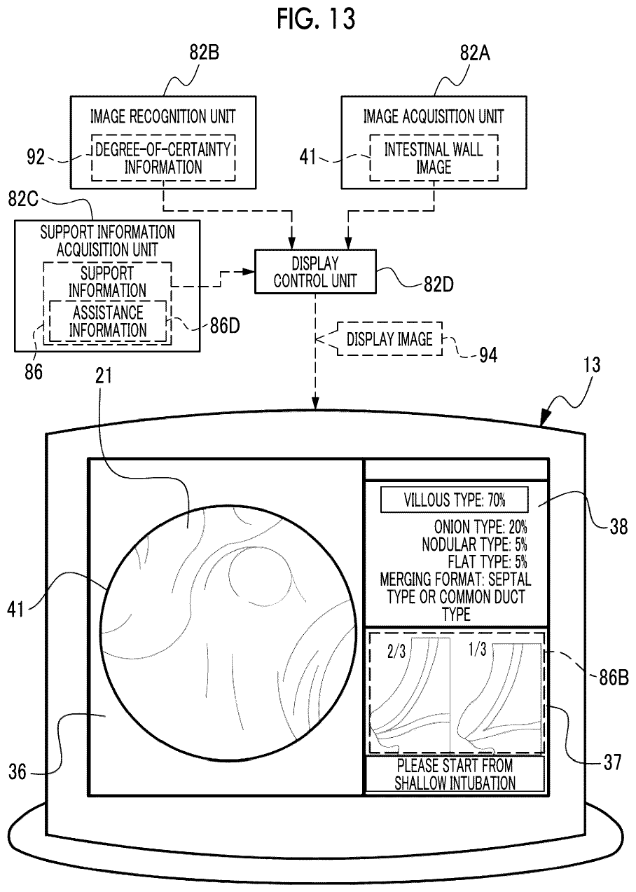

As shown in FIG. 13 as an example, the display control unit 82D acquires the intestinal wall image 41 from the image acquisition unit 82A. In addition, the display control unit 82D acquires the degree-of-certainty information 92 from the image recognition unit 82B. Moreover, the display control unit 82D acquires the support information 86 from the support information acquisition unit 82C. The support information 86 includes assistance information 86D. The assistance information 86D is information for assisting with the medical treatment, and the medical treatment here is a treatment performed for the merging format of the bile duct and the pancreatic duct determined according to the type of the papilla N.

For example, in a case where the type of the papilla N is the villous type, the appearance frequency of the merging format of the bile duct and the pancreatic duct is ⅔ of the septal type and ⅓ of the common duct type. That is, a plurality of merging formats of the bile duct and the pancreatic duct are assumed, and it is difficult to uniquely determine the merging format. In a case where the merging formats are different from each other, it is also necessary to appropriately change a procedure such as how to insert the cannula. Therefore, in a case where a plurality of merging formats are specified as being present, the assistance information 86D is provided to assist with the medical treatment, so that the user can easily perform the medical treatment. The assistance information 86D is an example of the “assistance information” according to the technology of the present disclosure.

The assistance information 86D may be set as, for example, an output value of the support information table 85 (see FIG. 11) or may be input in advance by the user. Examples of the content of the assistance indicated by the assistance information 86D include a content related to an insertion amount in a case where the cannula is inserted, a content related to the method of insertion, and the like.

The display control unit 82D generates a display image 94 including the intestinal wall image 41, the degree of certainty indicated by the degree-of-certainty information 92, and an assistance content indicated by the assistance information 86D, and displays the screen 36 to the screen 38 on the display device 13. A message indicating the assistance content is displayed on the screen 37 together with the schema 86B. In the example shown in FIG. 13, a message “Please start from shallow intubation” is displayed as the assistance content.

As described above, in the duodenoscope system 10 according to the present first modification example, the support information 86 includes the assistance information 86D, and the assistance information 86D is information for assisting with the medical treatment performed for the merging format determined according to the type of the papilla N. The assistance information 86D is output from the support information acquisition unit 82C. The message of the assistance content indicated by the support information 86 is displayed on the display device 13. The user can grasp the type of the papilla N and the assistance contents available for the medical treatment for the merging format while operating the duodenoscope 12. Accordingly, it is possible to contribute to the accurate implementation of the medical treatment for the merging format determined according to the type of the papilla N.

In addition, in the duodenoscope system 10 according to the present first modification example, in a case where a plurality of the merging formats of the bile duct and the pancreatic duct according to the type of the papilla N are present, the support information acquisition unit 82C of the processor 82 outputs the assistance information 86D. Accordingly, even in a case where the plurality of merging formats are present for the type of the papilla N, it is possible to contribute to the accurate implementation of the medical treatment for the merging format.

### Second Modification Example

In each of the above-described embodiments, although the form example in which the image recognition processing is performed on the entire intestinal wall image 41 to specify the type of the papilla N has been described, the technology of the present disclosure is not limited to this. In the present second modification example, type specification processing is performed after the papilla detection processing is performed on the intestinal wall image 41.

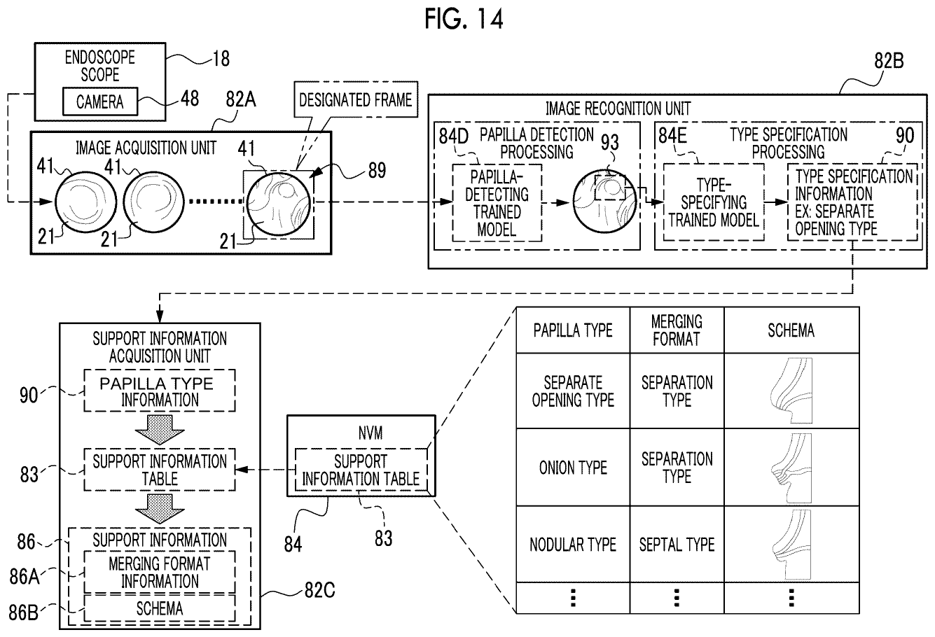

As shown in FIG. 14 as an example, the image acquisition unit 82A acquires the intestinal wall image 41 from the camera 48 provided in the endoscope scope 18. The image recognition unit 82B performs image recognition processing on the intestinal wall image 41. The image recognition processing includes papilla detection processing that is processing of detecting a region indicating the papilla N in the intestinal wall image 41, and type specification processing that is processing of specifying the type of the papilla N. The papilla detection processing is an example of the “first image recognition processing” according to the technology of the present disclosure, and the type specification processing is an example of the “second image recognition processing” according to the technology of the present disclosure.

First, the image recognition unit 82B performs the papilla detection processing on the intestinal wall image 41. The image recognition unit 82B inputs the intestinal wall image 41 acquired from the image acquisition unit 82A to a papilla-detecting trained model 84D. Accordingly, the papilla-detecting trained model 84D outputs papilla region information 93 corresponding to the input intestinal wall image 41. The papilla region information 93 is information (for example, position coordinates of a region indicating the papilla N in an image) capable of specifying a region indicating the papilla N in the intestinal wall image 41. The image recognition unit 82B acquires the papilla region information 93 output from the papilla-detecting trained model 84D.

The papilla-detecting trained model 84D is obtained by performing machine learning using training data on a neural network to optimize the neural network. Examples of the training data include training data in which a plurality of images (for example, a plurality of images corresponding to a plurality of time-series intestinal wall images 41) obtained in time series by imaging a part (for example, an inner wall of the duodenum) that can be a target for the ERCP examination are used as example data, and the papilla region information 93 is used as correct answer data.

The image recognition unit 82B performs the type specification processing on the region of the papilla N indicated by the papilla region information 93. The image recognition unit 82B inputs an image showing the papilla N specified by the papilla detection processing to a type-specifying trained model 84E. Accordingly, the type-specifying trained model 84E outputs the papilla type information 90 based on the input image showing the papilla N. The image recognition unit 82B acquires the papilla type information 90 output from the type-specifying trained model 84E.

The type-specifying trained model 84E is obtained by performing machine learning using training data on a neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is an annotation capable of specifying the type of the papilla N.

In addition, here, although the form example in which the papilla N is detected by using the papilla-detecting trained model 84D and the type of the papilla N is specified by using the type-specifying trained model 84E has been described, the technology of the present disclosure is not limited to this. For example, one trained model that performs the detection of the papilla N and the specification of the type of the papilla N on the intestinal wall image 41 may be used.

The support information acquisition unit 82C acquires the support information 86 corresponding to the type of the papilla N. The display control unit 82D (see FIG. 7) generates a display image 94 including the intestinal wall image 41, the type of the papilla N indicated by the papilla type information 90, and the merging format and the schema 86B indicated by the support information 86, and outputs the display image 94 to the display device 13.

As described above, in the duodenoscope system 10 according to the present second modification example, the image recognition processing is performed in the image recognition unit 82B of the processor 82. The image recognition processing includes the papilla detection processing and the type specification processing. Accordingly, since the type of the papilla N is specified for the papilla N specified by the papilla detection processing, the accuracy of specifying the type of the papilla N is improved compared to a case where the type specification processing is performed on the entire intestinal wall image 41.

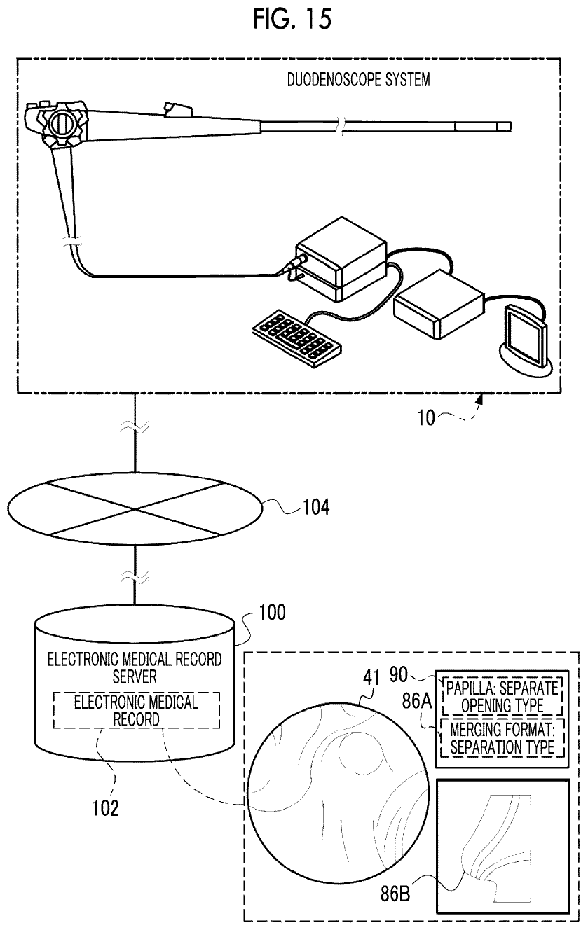

In each of the above embodiments, although the form example in which the papilla type information 90, the support information 86, the intestinal wall image 41, and the like are output to the display device 13, and these pieces of information are displayed on the screens 36 to 38 of the display device 13 has been described, the technology of the present disclosure is not limited to this. As shown in FIG. 15 as an example, the papilla type information 90, the support information 86, the intestinal wall image 41, and the like may be output to an electronic medical record server 100. The electronic medical record server 100 is a server that stores electronic medical record information 102 indicating a medical care result for a patient. The electronic medical record information 102 includes the papilla type information 90, the support information 86, the intestinal wall image 41, and the like.

The electronic medical record server 100 is connected to the duodenoscope system 10 via a network 104. The electronic medical record server 100 acquires the intestinal wall image 41 from the duodenoscope system 10. The electronic medical record server 100 stores the papilla type information 90, the support information 86, the intestinal wall image 41, and the like as some of the medical care results indicated by the electronic medical record information 102. The electronic medical record server 100 is an example of the “external device” according to the technology of the present disclosure, and the electronic medical record information 102 is an example of the “medical record” according to the technology of the present disclosure.

The electronic medical record server 100 is also connected to a terminal (for example, a personal computer installed in a medical care facility) other than the duodenoscope system 10 via the network 104. The user, such as the doctor 14, can obtain the papilla type information 90, the support information 86, the intestinal wall image 41, and the like stored in the electronic medical record server 100 via a terminal. In this way, since the papilla type information 90, the support information 86, the intestinal wall image 41, and the like are stored in the electronic medical record server 100, the user can obtain the papilla type information 90, the support information 86, the intestinal wall image 41, and the like.

In addition, in each of the above embodiments, although the form example in which the papilla type information 90, the support information 86, the intestinal wall image 41, and the like are output to the display device 13 has been described, the technology of the present disclosure is not limited to this. For example, the papilla type information 90, the support information 86, the intestinal wall image 41, and the like may be output to a voice output device such as a speaker (not shown), or may be output to a printing device such as a printer (not shown).

In addition, in each of the above embodiments, although the form example in which the image recognition processing using the AI method is executed on the intestinal wall image 41 has been described, the technology of the present disclosure is not limited to this. For example, image recognition processing using a pattern matching method may be executed.

In the above embodiment, although the form example in which the medical support processing is performed by the processor 82 of the computer 76 included in the image processing device 25 has been described, the technology of the present disclosure is not limited to this. For example, the medical support processing may be performed by the processor 70 of the computer 64 included in the control device 22. In addition, a device that performs the medical support processing may be provided outside the duodenoscope 12. An example of the device provided outside the duodenoscope 12 is at least one server and/or at least one personal computer that is communicably connected to the duodenoscope 12. In addition, the medical support processing may be performed in a distributed manner by a plurality of devices.

In the above embodiment, although the form example in which the medical support processing program 84A is stored in the NVM 84 has been described, the technology of the present disclosure is not limited to this. For example, the medical support processing program 84A may be stored in a portable non-transitory storage medium such as an SSD or a USB memory. The medical support processing program 84A stored in the non-transitory storage medium is installed in the computer 76 of the duodenoscope 12. The processor 82 performs the medical support processing according to the medical support processing program 84A.

In addition, the medical support processing program 84A may be stored in a storage device of, for example, another computer or a server connected to the duodenoscope 12 via a network. Then, the medical support processing program 84A may be downloaded and installed in the computer 76 in response to a request from the duodenoscope 12.

In addition, it is not necessary to store all of the medical support processing program 84A in the storage device of another computer or server connected to the duodenoscope 12, or the NVM 84, and part of the medical support processing program 84A may be stored.

Various processors described below can be used as the hardware resource for executing the medical support processing. An example of the processor is a CPU which is a general-purpose processor that executes software, that is, a program, to function as the hardware resource performing the medical support processing. In addition, an example of the processor includes a dedicated electronic circuit, which is a processor having a dedicated circuit configuration designed to perform specific processing, such as an FPGA, a PLD, or an ASIC. Any processor has a memory built in or connected to it, and any processor executes the medical support processing by using the memory.

The hardware resource for performing the medical support processing may be configured by one of the various processors or by a combination of two or more processors of the same type or different types (for example, a combination of a plurality of FPGAs or a combination of a CPU and an FPGA). The hardware resource for executing the medical support processing may also be one processor.

A first example of the configuration using one processor is a form in which one processor is configured by combining one or more CPUs and software, and the processor functions as the hardware resource for executing the medical support processing. A second example of the configuration is an aspect in which a processor that implements the functions of the entire system including a plurality of hardware resources for performing the medical support processing using one IC chip is used. A representative example of this aspect is an SoC. In this way, the medical support processing is implemented by using one or more of the various processors as the hardware resource.

More specifically, an electric circuit in which circuit elements such as semiconductor elements are synthesized can be used as a hardware structure of the various processors. In addition, the above medical support processing is merely an example. Therefore, it goes without saying that unnecessary steps may be deleted, new steps may be added, and the processing order may be changed within a range that does not deviate from the scope.

The above described contents and shown contents are detailed descriptions of portions relating to the technology of the present disclosure and are merely examples of the technology of the present disclosure. For example, the description of the configuration, the function, the operation, and the effect above are the description of examples of the configuration, the function, the operation, and the effect of the portions according to the technology of the present disclosure. As a result, it goes without saying that unnecessary parts may be deleted, new elements may be added, or replacements may be made with respect to the above-described contents and the above-shown contents within a range that does not deviate from the gist of the technology of the present disclosure. In addition, the description of, for example, common technical knowledge that does not need to be particularly described to enable the implementation of the technology of the present disclosure is omitted in the above described contents and shown contents in order to avoid confusion and to facilitate understanding of the portions relating to the technology of the present disclosure.

In the present specification, “A and/or B” is synonymous with “at least one of A or B” That is, “A and/or B” may mean only A, only B, or a combination of A and B. In the present specification, the same concept as “A and/or B” also applies to a case in which three or more matters are expressed by association with “and/or”.

All documents, patent applications, and technical standards described in the present specification are incorporated in the present specification by reference in their entirety to the same extent as in a case where the individual documents, patent applications, and technical standards are specifically and individually written to be incorporated by reference.

The disclosure of JP2022-177612 filed on Nov. 4, 2022, is incorporated in the present specification by reference.

What is claimed is:

1. A medical support device comprising:
a processor,
wherein the processor is configured to:
specify a papilla type, which is a type of a duodenal papilla, by executing image recognition processing on an intestinal wall image obtained by imaging an intestinal wall including the duodenal papilla in a duodenum with a camera provided in an endoscope scope; and
output related information related to the papilla type,
wherein the related information is information depending on the specified papilla type.

2. The medical support device according to claim 1,
wherein the outputting of the related information is displaying the related information on a screen.

3. The medical support device according to claim 1,
wherein the related information includes a schema determined according to the papilla type.

4. The medical support device according to claim 1,
wherein the related information includes merging format information, and
the merging format information is information that is determined according to the papilla type and that is capable of specifying a merging format in which a bile duct and a pancreatic duct merge with each other.

5. The medical support device according to claim 1,
wherein the image recognition processing includes classification processing of classifying the papilla types, and
the related information includes degree-of-certainty information indicating a degree of certainty for each papilla type classified by the classification processing.

6. The medical support device according to claim 1,
wherein an appearance frequency of a merging format in which a bile duct and a pancreatic duct merge with each other is determined for each papilla type, and
the processor is configured to output, as the related information, information including appearance frequency information indicating the appearance frequency according to the specified papilla type.

7. The medical support device according to claim 1,
wherein the papilla type includes a first papilla type,
the first papilla type has any one of a plurality of merging formats in which a bile duct and a pancreatic duct merge with each other, and
the processor is configured to output, in a case where the first papilla type is specified as the papilla type, information including appearance frequency information indicating an appearance frequency for each merging format as the related information.

8. The medical support device according to claim 7,
wherein the first papilla type is a villous type or a flat type, and
the plurality of merging formats are a septal type and a common duct type.

9. The medical support device according to claim 1,
wherein the related information includes assistance information, and
the assistance information is information for assisting with a medical treatment performed for a merging format in which a bile duct and a pancreatic duct merge with each other and which is determined according to the papilla type.

10. The medical support device according to claim 9,
wherein the processor is configured to output the assistance information in a case where a plurality of the merging formats are present in the specified papilla type.

11. The medical support device according to claim 1,
wherein the processor is configured to specify the papilla type by executing the image recognition processing on the intestinal wall image in units of frames.

12. The medical support device according to claim 1,
wherein the image recognition processing includes first image recognition processing and second image recognition processing, and
the processor is configured to:
detect a duodenal papilla region by executing the first image recognition processing on the intestinal wall image; and
specify the papilla type by executing the second image recognition processing on the detected duodenal papilla region.

13. The medical support device according to claim 1,
wherein the related information is stored in an external device and/or a medical record.

14. An endoscope comprising:
the medical support device according to claim 1; and
the endoscope scope.

15. A medical support method comprising:
specifying a papilla type, which is a type of a duodenal papilla, by executing image recognition processing on an intestinal wall image obtained by imaging an intestinal wall including the duodenal papilla in a duodenum with a camera provided in an endoscope scope; and
outputting related information related to the papilla type,
wherein the related information is information depending on the specified papilla type.

16. A non-transitory computer-readable storage medium storing a program executable by a computer to execute processing comprising:
specifying a papilla type, which is a type of a duodenal papilla, by executing image recognition processing on an intestinal wall image obtained by imaging an intestinal wall including the duodenal papilla in a duodenum with a camera provided in an endoscope scope; and
outputting related information related to the papilla type,
wherein the related information is information depending on the specified papilla type.
